(19) United States
(12) Patent Application Publication
(10) Pub. No.: US 2015/0359949 A1
(43) Pub. Date: Dec. 17, 2015

(54) Polyp Filter

(71) Applicant: Medline Industries, Inc., Mundelein, IL (US)

(72) Inventors: Anna Yeager, Chicago, IL (US); James Burgess, Mundelein, IL (US); Scott Bedoe, McHenry, IL (US)

(21) Appl. No.: 14/306,827

(22) Filed: Jun. 17, 2014

(51) Int. Cl. A61M 1/00

(52) U.S. Cl. CPC A61M 1/0056

(57) ABSTRACT

A polyp filter is non-integral to a corresponding suction-line dedicated polyp track. By one approach this polyp filter has a threaded portion that threadably engages at least one of a suction-line dedicated polyp trap housing and lid. That threaded portion can comprise, for example, a threaded hole that is formed axially through the polyp filter. By one approach that threaded portion threadably engages a threaded tube as comprises a part of the lid.

### TECHNICAL FIELD

These teachings relate generally to polyp traps.

### BACKGROUND

A polyp is an abnormal growth of tissue projecting from a mucous membrane. Polyps are commonly found in the colon, stomach, nose, sinus(es), urinary bladder, and uterus though they can occur elsewhere in the body where mucous membranes exist. Colon polyps are a concern because of the corresponding potential for colon cancer. Polyps are often discovered at the time of screening (for example, during a colonoscopy) and removed endoscopically.

Polyps so removed typically exit the patient's body via a suction tube. That suction tube typically connects to an in-line polyp trap that is located between the endoscope and the suction mechanism. These polyp traps typically contain one or more filters that serve to separate removed polyps from corresponding endoscopic effluent.

The aforementioned filters are typically an integral part of either the polyp trap housing or the lid for the polyp trap housing. Though typically efficacious in use, existing polyp trap designs tend to be relatively complex structures, somewhat difficult to design and manufacture, and hence relatively expensive.

### BRIEF DESCRIPTION OF THE DRAWINGS

The above needs are at least partially met through provision of the polyp filter described in the following detailed description, particularly when studied in conjunction with the drawings, wherein:

FIG. 1 comprises a top perspective view as configured in accordance with various embodiments of these teachings;

FIG. 2 comprises a bottom perspective view as configured in accordance with various embodiments of these teachings;

FIG. 3 comprises an exploded bottom perspective view as configured in accordance with various embodiments of these teachings;

FIG. 4 comprises a bottom perspective view as configured in accordance with various embodiments of these teachings;

FIG. 5 comprises a top perspective view as configured in accordance with various embodiments of these teachings;

FIG. 6 comprises a side elevational view as configured in accordance with various embodiments of these teachings; and

FIG. 7 comprises a top perspective view as configured in accordance with various embodiments of the invention.

Elements in the figures are illustrated for simplicity and clarity but are drawn to scale. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments a polyp filter is non-integral to a corresponding suction-line dedicated polyp trap. By one approach this polyp filter has a threaded portion that threadably engages at least one of a suction-line dedicated polyp trap housing and lid (as used herein, this reference to a “dedicated” polyp trap housing and lid is meant to clarify that the housing and lid are designed and provided to the end-user specifically as a polyp trap housing and lid and not as a more generic trap mechanism for other biological materials). That threaded portion can comprise, for example, a threaded hole that is formed axially through the polyp filter. By one approach that threaded portion threadably engages a threaded tube as comprises a part of the lid.

So configured, the polyp filter can move between a removed position and a fully-installed position by rotating about the aforementioned threaded portion with respect to the lid. By one approach the polyp filter has an external periphery that fully engages an internal surface of the housing when in a fully-installed position with respect to the housing to thereby form a seal at the external periphery.

Accordingly, pursuant to these teachings, a polyp filter constitutes a separate, discrete, physical component as compared to either the polyp trap housing or lid. So configured, the polyp filter can be separately packaged in end-user packaging from either of the housing or the lid. Regardless of what packaging preferences are observed, these teachings offer an opportunity for simplified manufacturing designs and hence an opportunity to reduce cost. Also, these teachings readily permit more than one polyp filter to be used in temporal series with a single polyp trap when trapping a series of removed polyps for a particular patient. Such an approach represents reduced waste and again an opportunity to reduce cost.

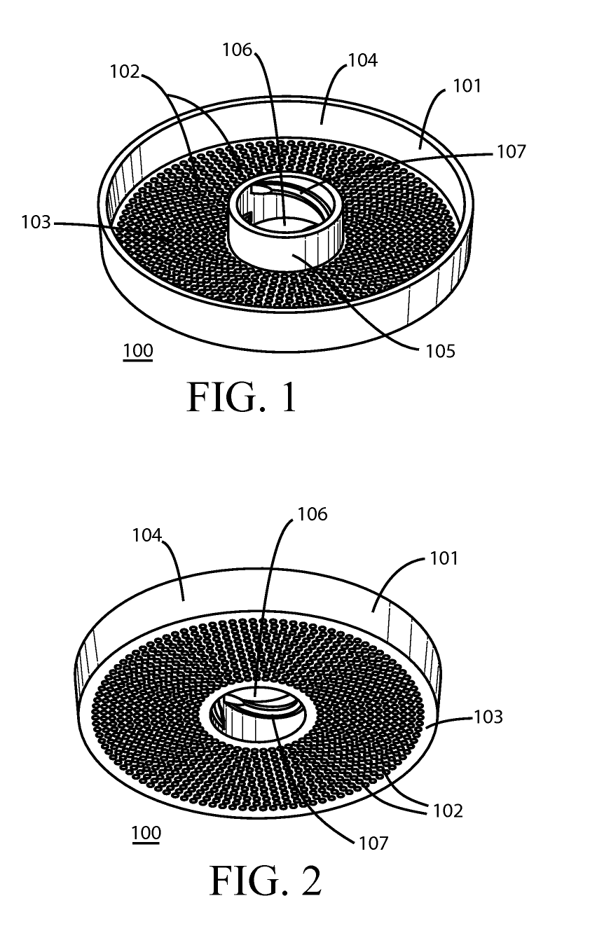

These and other benefits may become clearer upon making a thorough review and study of the following detailed description. Referring now to the drawings, FIGS. 1 and 2 present an illustrative example of a polyp filter 100 that accords with these teachings This polyp filter 100 comprises, in this example, a circular shallow dish 101 having a plurality of filter openings 102 disposed through a bottom portion 103 thereof. In this example the filter openings 102 are very small as compared to the likely size of any polyps to be captured and hence most if not all polyps are unlikely to pass through the filter 100. Corresponding effluent, however, can and will pass through these openings 102.

A wall 104 completely encircles the bottom portion 103. The outside surface of this wall 104 therefore constitutes an external periphery for the polyp filter 100. The polyp filter 100 also includes, in this example, a short tube 105 extending upwardly from the center of the bottom portion 103. This short tube 105 has a hole 106 disposed axially therethrough to serve as a pneumatic pathway. The interior portion of this tube 105 comprises a threaded portion in this example and therefore includes a thread 107 formed thereon. The length, width, and pitch of this thread 107 can vary as desired.

So configured, this polyp trap is non-integral with respect to any other parts of a corresponding polyp trap including a polyp trap housing or a polyp trap lid. As used herein, this reference to being non-integral will be understood to mean that the polyp filter is not of a piece with those other components via, for example, co-molding or the like. To look at this another way, “integral” will be understood to refer to a combination and joinder that is sufficiently complete so as to consider the combined elements to be as one. Accordingly, two items would not be considered “integral” with respect to one another if they are merely connected to one another by the action of a holding member such as a screw, bolt, clamp, clip, or the like.

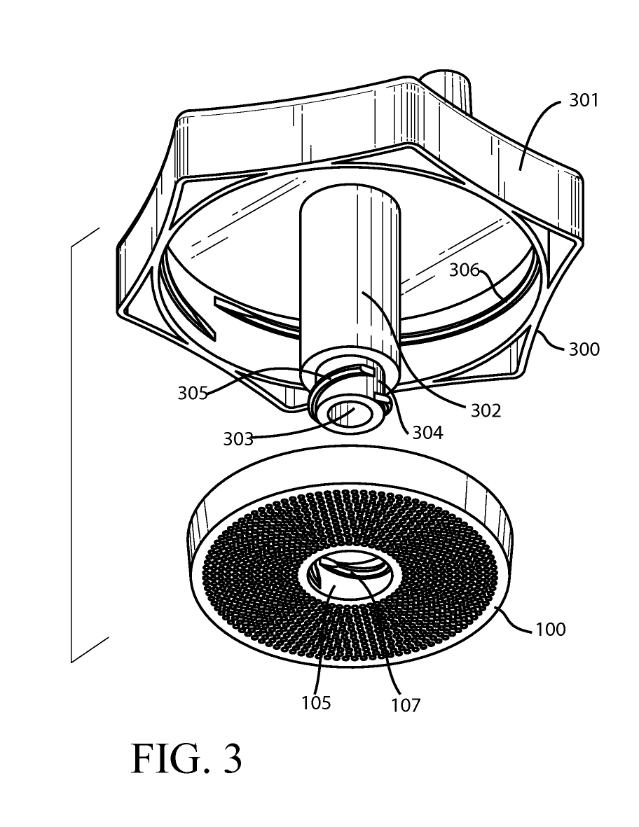

Referring now to FIG. 3, although the polyp filter is non-integral to either a polyp trap housing or lid, in this illustrative example the polyp filter is nevertheless configured to be selectively attached to the polyp trap lid 300. This polyp trap lid 300 has a contoured exterior surface 301 to facilitate its being grasped and manipulated by a medical services provider's hand. A thread 306 is disposed on the interior surface thereof to facilitate screwing the lid 300 on to a corresponding polyp trap housing as described below.

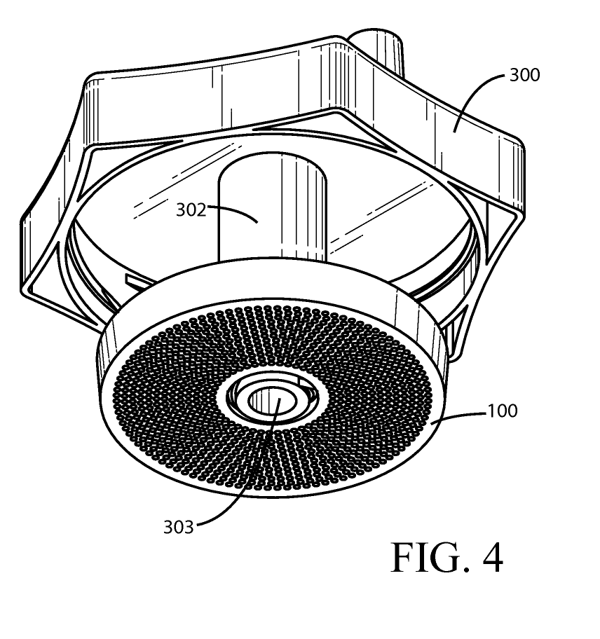

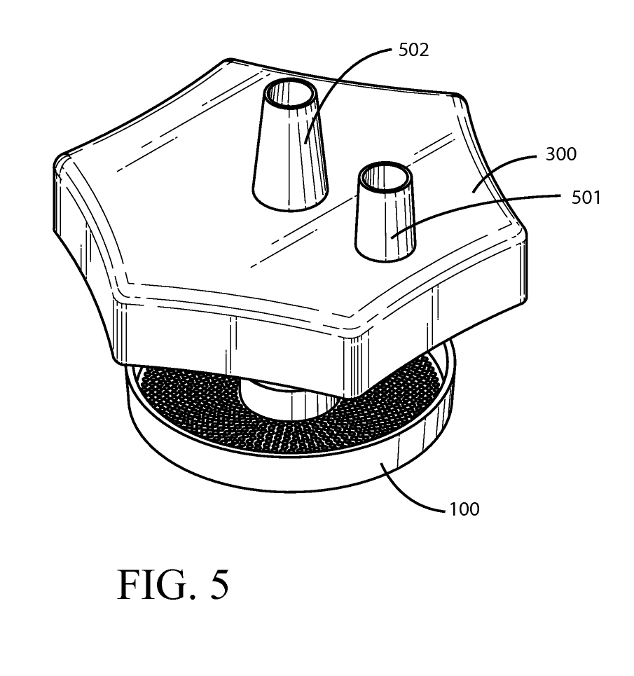

In this illustrative example the lid 300 has a tube 302 extending outwardly therefrom. This tube 302 has a hollow interior 303 that serves as a pneumatic pathway when placing the polyp trap in-line with a suction system. The end portion 304 of this tube 302 has a thread 305 disposed thereabout. This thread 305 is configured to compatibly mate with the thread 107 of the polyp filter 100. Accordingly, and as shown in FIG. 4 and FIG. 5, the polyp filter 100 can threadably engage the aforementioned threaded portion of the lid 300 to thereby move from a removed position with respect to the lid 300 to a fully-installed position with respect to the lid 300.

With particular reference to FIG. 5, the lid 300 also includes two suction tube interfaces 501 and 502. An eccentrically positioned one of these suction tube interfaces 501 connects to an endoscopic tool via a corresponding length of pneumatic tubing. An axially-located suction tube interface 502 pneumatically couples to the pathway 303 formed by the aforementioned tube 302 and serves to connect to the suction system via another corresponding length of pneumatic tubing.

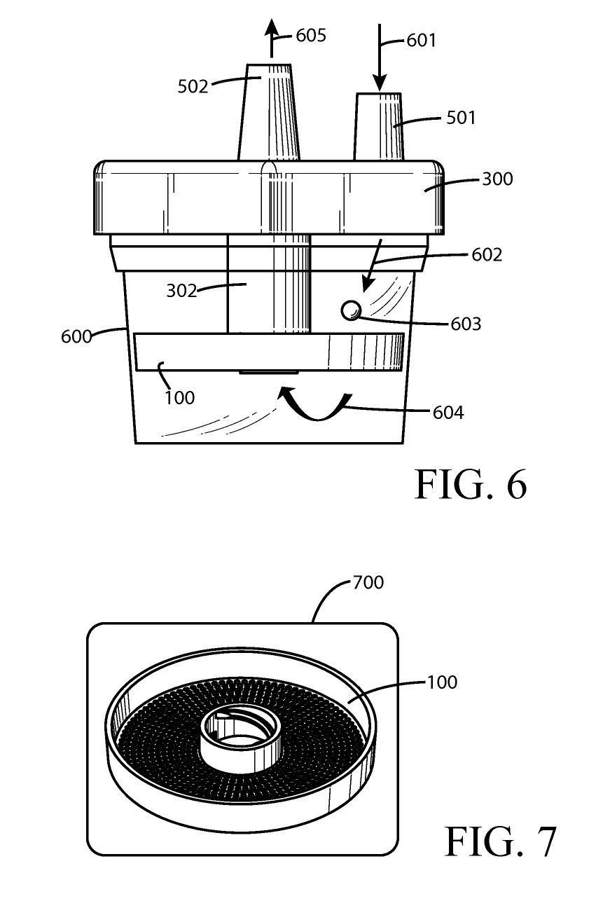

So configured, and as illustrated in FIG. 6, endoscopic effluent and one or more separated polyps can enter the polyp trap lid 300 as represented by the arrow denoted by reference numeral 601. When the polyp trap lid 300 is attached to a corresponding polyp trap housing 600 (the latter essentially comprising a cup or canister), that material then enters the polyp trap housing 600 as represented by the arrow denoted by reference numeral 602. In this example that material includes a polyp 603. The polyp trap filter 100 prevents the polyp 603 from traveling further into the polyp trap housing 600 while allowing the effluent material to pass therethrough as represented by the arrow denoted by reference numeral 604. That material then passes up through the lid tube 302 such that that material exits the polyp trap and continues on towards the suction system as represented by the arrow denoted by reference numeral 605.

In this illustrative example the aforementioned external periphery of the polyp filter 100 fully engages an internal surface of the housing 600 to thereby form a seal at the external periphery. (In the illustration provided there is a small gap between that external periphery of the filter 100 and the housing sidewall 600. For the sake of clarity and simplicity the internal wall of the housing sidewall 600 is not shown, hence creating the illusion of that gap.) This seal ensures that a polyp cannot avoid being captured by the filter 100 by sliding between the filter 100 and the housing 600. If desired, however, useful results may nevertheless be attained even if a small opening exists between the external periphery of the filter 100 and the internal sidewall of the housing 600.

So configured, such a filter 100 can be quickly, easily, and intuitively securely attached to the polyp trap lid 300. The lid 300 can then be threadably attached to a corresponding polyp trap housing 600 and the resultant polyp trap thereby readied for use. Notwithstanding this convenience of use, such components are each readily manufactured (employing, for example, any of a variety of plastics and corresponding blow molding techniques).

During use, the described configuration will also readily support changing the filter with each trapped polyp without also requiring disconnecting and reconnecting the suction system from the lid 300 and without requiring a new lid 300 or housing 600. This capability can save considerable time when supporting such a protocol as well as incurring reduced costs with respect to required materials.

In part because a single polyp trap may be conveniently and effectively used with a plurality of such filters 100 during course of use with a single patient, it may be useful to individually package these filters 100 in a singulated manner. For example, as shown in FIG. 7, such a polyp filter 100 can be wrapped in end-user packaging 700 separately from either of the housing 600 and the lid 300. (As used herein, the expression “end-user packaging” will be understood to refer to packaging that is designed and intended during ordinary use to be opened by the person who will typically be using the filter, such as a medical services provider.)

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. A suction-line dedicated polyp trap having a non-integral polyp filter.

2. The suction-line dedicated polyp trap of claim 1 further comprising a housing configured to receive the non-integral polyp filter.

3. The suction-line dedicated how a trap of claim 2 further comprising a lid configured to connect to the housing.

4. The suction-line dedicated polyp trap of claim 3 wherein the lid includes a threaded portion.

5. The suction-line dedicated polyp trap of claim 4 wherein the non-integral polyp filter includes a threaded portion configured to threadably engage the threaded portion of the lid.

6. The suction-line dedicated polyp trap of claim 3 wherein the non-integral polyp filter is configured to move between a fully-installed position and a removed position with respect to the lid.

7. The suction-line dedicated polyp trap of claim 6 wherein the non-integral polyp filter has an external periphery that fully engages an internal surface of the housing when in the fully-installed position to thereby form a seal at the external periphery.

8. The suction-line dedicated polyp trap of claim 6 wherein the non-integral polyp filter includes a threaded portion and moves between the removed position and the fully-installed position by rotating about the threaded portion with respect to the lid.

9. The suction-line dedicated polyp trap of claim 5 wherein the non-integral polyp filter comprises a shallow dish having a plurality of filter openings disposed through a bottom portion thereof.

10. The suction-line dedicated polyp trap of claim 9 wherein the threaded portion of the non-integral polyp filter comprises a threaded hole formed through the bottom portion of the shallow dish.

11. The suction-line dedicated polyp trap of claim 3 wherein the non-integral polyp filter is wrapped in end-user packaging separately from either of the housing and the lid.

12. A polyp filter having a threaded portion for threadably engaging at least one of a suction-line dedicated polyp trap housing and lid.

13. The polyp filter of claim 12 wherein the threaded portion comprises a threaded hole formed axially through the polyp filter.

14. The polyp filter of claim 13 wherein the threaded portion of the polyp filter is configured to threadably engage a threaded tube as comprises a part of the lid.

15. The polyp filter of claim 14 wherein the threaded tube comprises a suction line return path during ordinary use of the suction-line dedicated polyp trap housing.

16. The polyp filter of claim 13 wherein the polyp filter further comprises a shallow dish having a plurality of filter openings disposed through a bottom portion thereof.

17. The polyp filter of claim 12 wherein the polyp filter is wrapped in end-user packaging separately from either of the suction-line dedicated polyp trap housing and the lid.
